(19) United States
(12) Patent Application Publication
(10) Pub. No.: US 2013/0035578 A1
(43) Pub. Date: Feb. 7, 2013

(54) Portable Brain Activity Monitor and Method

(71) Applicants: Gordon Chiu, Chatham, NJ (US); Larry Douglas Reid, Troy, NY (US)

(72) Inventors: Gordon Chiu, Chatham, NJ (US); Larry Douglas Reid, Troy, NY (US)

(21) Appl. No.: 13/563,092

(22) Filed: Jul. 31, 2012

Related U.S. Application Data
(60) Provisional application No. 61/513,763, filed on Aug. 1, 2011.

(51) Int. Cl. A61B 5/0478; A61B 5/0484

(52) U.S. Cl. 600/379

(57) ABSTRACT

The current invention discloses a portable brain-activity monitor for recording a subject's EEG while he/she is listening to music. The brain-activity monitor includes an earphone connected to a portable audio player as well as an EEG sensor. The EEG sensor is connected to EEG electrodes that are shaped and sized to fit within the auditory canal. Suitable electronic processing circuitry, located in close proximity to the electrode, amplify the EEG signals in a range of 0.5 Hz to 100 Hz, perform an analogue-to-digital conversion, filter the output and deliver it to a recording device. A cable connected to the subjects earlobe provides the ground for the EEG sensor. The monitor is preferably Bluetooth enabled to allow it to connect to computing devices such as PCs and smart phones.

### CLAIM OF PRIORITY

This utility application claims priority from U.S. provisional application 61/513,763 filed on Aug. 1, 2011, the content of which is fully incorporated herein.

### FIELD OF THE INVENTION

The invention relates to a method and device for monitoring mammalian brain EEG during normal activity, and more particularly to a portable device and related methods to monitor brain EEG in the mammalian brain via the ear canal, while music is played to the mammal whose EEG is being monitored.

### BACKGROUND OF THE INVENTION

“Music has charms to soothe a savage breast”. William Congreve (1697)

Although it has long been apparent that music can have a marked effect on the emotional state of a listener, it remains unclear how much of that effect is due to inherent qualities of the music, and how much may be ascribed to “anchoring” effects in which stimuli trigger emotions with which they have a prior association.

One clinically proven method of monitoring the state of a person's brain is Electroencephalography, commonly known as EEG. EEG is the recording of the brain's electrical activity via electrodes that are typically placed on the subject's scalp. EEG recordings usually comprise two components: transients and rhythmic activity. In the years since the pioneering work of Hans Berger, who recorded the first human EEG in 1924, the frequency of the rhythmic EEG component has been shown to be closely associated with particular states of mental activity of the subject. For instance, rhythmic activity with a frequency of 8-12 Hz, so called Alpha waves, have been shown to be indicative of a relaxed state of mind, while rhythmic activity with a frequency of 13-30 Hz, so called Beta waves, have been shown to be indicative of a state of the subject being alert with active concentration.

EEG recording devices have also changed over the years, benefiting from improvements in electrical equipment such as, but not limited to, more sensitive differential amplifiers, better electrodes and miniaturization of components. These advances, and their corresponding reductions in cost, have lead to EEGs being used for many purposes besides conventional clinical diagnosis and cognitive neuroscience. One important extension of use of EEG equipment is in neurofeedback. In its most advanced form, neurofeedback is being used for brain computer interfaces. For instance, in 2007 NeuroSky released the first affordable consumer based EEG game controller, along with the game NeuroBoy™. This was also the first large scale EEG device to use dry sensor technology.

Despite all the improvements, EEG recording devices tend to require multiple electrodes electrically connected to the scalp and functionally connected to the associated electronics by a multiplicity of wires.

For recording an EEG during a subject's routine activities, including listening to music, a more discrete device is preferred. The device of the current invention is such a device, being designed to record with a minimal number of electrodes that may be concealed within the subject's audio canal, and in which the initial recording electronics is either concealed with the audio canal on located in a discrete earphone device. In a preferred embodiment, the brain activity monitor of this invention may be blue tooth enabled, allowing relay of the detected data to a recording device without the need for further wires.

### DESCRIPTION OF THE RELATED ART

The relevant prior art includes:

U.S. Patent Application 20050197590 by I. Osorio published on Sep. 8, 2005 entitled “System for the prediction, rapid detection, warning, prevention, or control of changes in activity states in the brain of a subject” that describes a system that analyzes signals representative of a subject's brain activity in a signal processor for information indicating the subject's current activity state and for predicting a change in the activity state. One preferred embodiment uses a combination of nonlinear filtering methods to perform real-time analysis of the electro-encephalogram (EEG) or electro-corticogram (ECoG) signals from a subject patient for information indicative of or predictive of a seizure, and to complete the needed analysis at least before clinical seizure onset. The preferred system then performs an output task for prevention or abatement of the seizure, or for recording pertinent data.

U.S. Patent Application no. 20110054240 by E. L. Bender published on Mar. 3, 2011 entitled “Induced Relaxation and Therapeutic Apparatus and Method” that describes an apparatus and method for introducing multisensory stimuli. The apparatus includes an ergonomically contoured seating device, at least one vibrating acoustic device, at least one plate for dispersing vibration throughout the entire seating device, a rotatable mechanism for rotating the seating device.

Various implements are known in the art, but fail to address all of the problems solved by the invention described herein. Two embodiments of this invention are illustrated in the accompanying drawings and will be described in more detail herein below.

### SUMMARY OF THE INVENTION

The present invention relates to a portable brain activity monitor, designed for recording EEG activity during routine functioning of a subject, including when the subject is listening to music or other audio programs. The device may be used on any mammalian subject, though a human is a preferred subject.

In one preferred embodiment, the brain activity monitor may include an earphone that may be functionally connected to a portable audio player. The earphone may be used to deliver music, or other audio content, to the subject.

The brain activity monitor may, for instance, have a low-noise EEG sensor. The low-noise EEG sensor may have one or more EEG electrodes that may be shaped and sized to fit within a mammalian auditory canal. The low-noise EEG sensor may also have suitable electronic processing circuitry located in close proximity to the EEG electrode.

In one preferred embodiment, the processing circuitry may have an analogue amplifier that may be designed to amplify electrical signals in a range of 0.5 Hz to 100 Hz. The processing circuitry may also have other electronic modules such as, but not limited to, an analogue-to-digital conversion circuit and a digital filtering circuit, or some combination thereof.

The brain activity monitor may also have an earth or grounding cable that may, for instance, be an ear attachment device. The earth cable and the low-noise sensor may both be functionally connected to the low-noise EEG sensor. The brain activity monitor may also be functionally connected to a portable digital electronic storage device.

The brain activity monitor may also include a power source, preferably a battery, and more preferably a rechargeable battery. The rechargeable battery can be recharged with a Universal Serial Bus (USB) circuit or other connections. The power source may provide power to record brain activities.

The brain activity monitor may also include a power signal as an indicator as to whether the power is on or off, and whether the power is sufficient or inadequate. The preferred form of power signal is a signaling light.

The brain activity monitor may also include a power switch and the user may turn the monitor on or off using the power switch. Since the brain activity monitor may include an earphone for playing music or other audio programs, the power switch may control not only the recording of EEG, but also the music and audio signals.

The brain activity monitor may include a casing, which may be used to house the various components of the device.

Therefore, the present invention succeeds in conferring the following, and others not mentioned, desirable and useful benefits and objectives.

It is an object of the present invention to provide a brain activity monitor that may be worn and operate during a subject's routine activities.

It is another object of the present invention to provide a brain activity monitor that has a visually discrete appearance when worn.

Yet another object of the present invention is to provide a brain activity monitor that records with a minimal number of electrodes.

Still another object of the present invention is to provide a brain activity monitor that records with electrodes that may be concealed within the subject's audio canal.

Yet another object of the present invention is to provide a brain activity monitor in which the initial recording electronics is either concealed with the audio canal or located in a discrete earphone device.

Still another object of the present invention is to provide a brain activity monitor that is BlueTooth® enabled.

Still another object of the present invention is to provide a brain activity monitor that is robust and may be used when a subject is participating in sporting events, including contact sports such as, but not limited to, football, lacrosse, boxing or ice hockey.

Yet another object of the present invention is to provide a brain activity monitor that is light and portable.

Still another object of the present invention is to provide a brain activity monitor that is easy to use and easy to manufacture.

Yet another object of the present invention is to provide a brain activity monitor that includes a power source such as a rechargeable battery.

Still another object of the present invention is to provide a brain activity monitor that may be used with mobile computing devices such as smart phones, tablet PC, and laptop computers.

Still another object of the present invention is to provide a brain activity monitor having an integrated earphone-electrode unit.

### BRIEF DESCRIPTION OF THE DRAWINGS

FIG. 1 shows a first preferred embodiment of the present invention being worn by a subject.

FIG. 2 shows a schematic cross-section of the first preferred embodiment of the present invention located within a subject's audio canal.

FIG. 3A shows a front perspective view of the second preferred embodiment of the present invention.

FIG. 3B shows a back perspective view of the second preferred embodiment of the present invention.

FIG. 3C shows a cross-sectional view of the second preferred embodiment of the present invention.

FIG. 4 shows a front perspective view of a third preferred embodiment of the present invention.

FIG. 5 shows a front perspective view of a single monitoring unit of the third preferred embodiment with the exchangeable EEG electrode-earphone combinations having different sizes.

FIG. 6 shows a front perspective view of the third preferred embodiment with more details.

FIG. 7A shows a first circuit diagram of the third preferred embodiment of the present invention.

FIG. 7B shows a second circuit diagram of the third preferred embodiment of the present invention.

FIG. 7C shows a third circuit diagram of the third preferred embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to embodiments of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

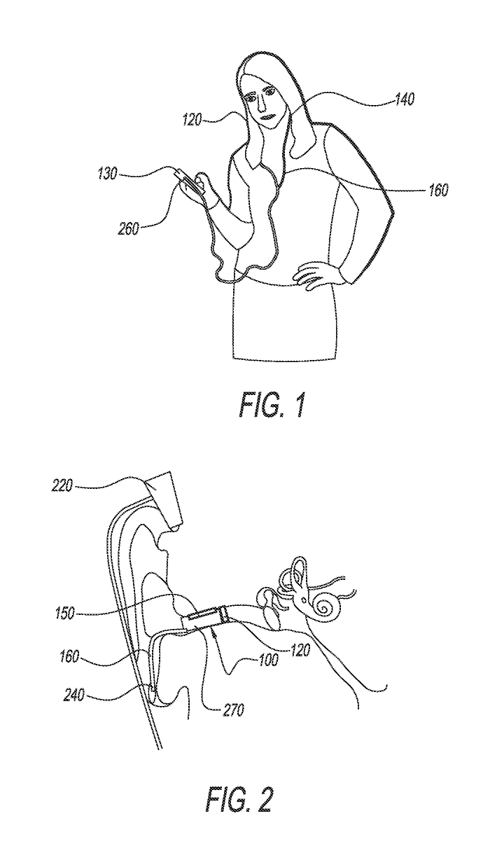

FIG. 1 schematically shows a first preferred embodiment of the brain activity monitor 100 of the present invention being worn by a subject. The subject may be any mammal, though in a preferred embodiment, the subject is an adult or child human.

In this embodiment, the brain activity monitor 100 may include an earphone 120, shown located in the subject's ear. The earphone 120 may, for instance, be used to supply the subject with music while the brain activity-monitor 100 records the subject's EEG to monitor the subject's mental activity state that the music elicits.

The earphone 120 may be functionally connected to a portable audio player 130, shown being held by the subject. The functional connection between the earphone 120 and the portable audio player 130 may be by any suitable means such as, but not limited to, a wired connection, an optical connection, a wireless connection, a WiFi connection or a BlueTooth® connection, or some combination thereof.

As shown in FIG. 1, the brain activity monitor 100 may have a low-noise EEG sensor 140 that may be located in the subject's other audio canal.

FIG. 2 shows a schematic cross-section of an embodiment of the brain activity monitor 100 located within the subject's auditory canal 270.

The low-noise EEG sensor 140 (as shown in FIG. 1) may include an EEG electrode 150 and an earth cable 160. The earth cable 160 may be functionally attached to the low-noise EEG sensor 140 so that the subject's EEG may be obtained by the low-noise EEG sensor 140. The EEG may be obtained by, for instance, monitoring the voltage between the EEG electrode 150 that may be electronically attached to the subject's skin within the subject's ear canal, and the earth, or grounding, cable 160 that may be removably, electronically attached to another area of the subject's skin such as, but not limited to, the subject's earlobe.

The low-noise sensor may also be functionally connected to a digital electronic storage device 260, as shown in FIG. 1. In FIG. 1 the digital electronic storage device 260 is shown as part of the portable audio player 130 being held by the subject. The digital electronic storage device 260 may be any suitable digital data storage device such as, but not limited to, a non-volatile memory card, a PC card, a USB flash drive or some combination thereof. The digital electronic storage device 260 may also refer to devices such as a desktop, a laptop, portable computing devices such as tablet PC and smart phones, including but not limited to iPhone® and Android® phones. The functional connection between the low-noise EEG sensor 140 and the portable digital electronic storage device 260 may be by any suitable means such as, but not limited to, a wired connection, an optical connection, a wireless connection, a WiFi connection or a BlueTooth connection, or some combination thereof.

The EEG electrode 150 of the brain activity monitor 100 is shown, in FIG. 2, in contact with the upper portion of the subject's auditory canal 270.

The embodiment of the brain activity monitor 100 shown in FIG. 2 has an earphone 120 incorporated into it, and may, therefore, both deliver audio to the subject and record the subject's EEG elicited in response to the delivered audio.

The earth cable 160 may be connected at one end to the brain activity monitor 100 and at the other end to an ear attachment device 240.

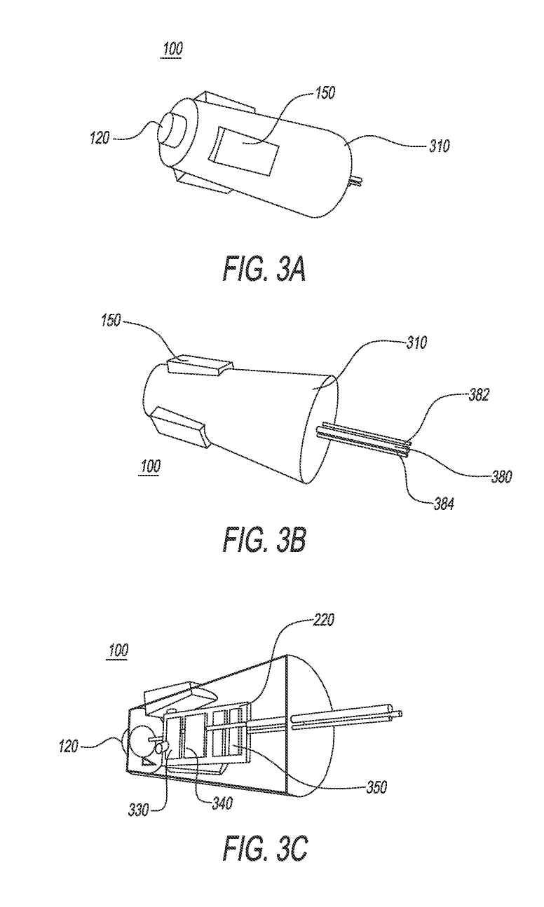

As is shown in FIG. 3C, he brain activity monitor 100 may be functionally attached to the electronic processing circuitry 220 that may be external to the auditory canal 270 and may, for instance, be located within close proximity to the EEG electrode 150 in an ear bud type earphone.

The electronic processing circuitry 220 may include processing circuitry that may include an analogue amplifier 330 designed to amplify electrical signals in a range of 0.5 Hz to 100 Hz, and an analogue-to-digital conversion circuit 340 and a digital filtering circuit 350.

FIG. 3A shows a front perspective view of a second preferred embodiment of the brain activity monitor 100.

In this embodiment of the brain activity monitor 100, the entire device may be shaped and sized to fit into an auditory canal 270. The brain activity monitor 100 may, for instance, be housed within an outer shell 310 that has the shape of a conical frustum. The outer shell 310 may also be made of, or coated with, a suitable deformable material such as, but not limited to, a foam or a memory foam. In this way the brain activity monitor 100 may be inserted into, and later removed from, the subject's auditory canal 270 in much the same way as an ear-plug.

The EEG electrodes 150 may protrude through the outer shell 310 so as to contact the auditory canal 270 when the brain activity monitor 100 is inserted into the auditory canal 270. The EEG electrode 150 may be any suitable electrode such as, but not limited to, a dry contact electrode, a non-gel, wettable surface electrode, a capacitance electrode or a silicon needle electrode or some combination thereof.

The earphone 120 may also protrude through the outer shell 310, but through the front so as not to touch the auditory canal 270 but to be able to deliver audio within the auditory canal 270.

FIG. 3B shows a back perspective view of the second preferred embodiment of the brain activity monitor 100.

This view shows that there may be three functional connections leading to the brain activity monitor 100. A common ground 380 may be used as the ground line for both the earphone audio signal 382 and the encoded EEG signal 384, though there may be reason to have separate ground lines for each.

FIG. 3C shows a cross-sectional view of the second preferred embodiment of the brain activity monitor 100.

The analogue amplifier 330 may be the first element connected to the EEG electrode 150. The analogue amplifier 330 may, for instance, include suitable differential amplifiers to amplify electrical signals in a range of 0.5 Hz to 100 Hz.

The analogue amplifier 330 may in turn be connected to the analogue-to-digital conversion circuit 340. The analogue-to-digital conversion circuit 340 may, for instance, may be an 18 bit AD convertor 370.

The analogue amplifier 330 may in turn be connected to the digital filtering circuit 350. The digital filtering circuit 350 may, for instance, include a 60 Hz notch filter 360 to ensure that no main current noise is included in the signal when sent out on the encoded EEG signal 384.

The brain activity monitor 100 of this embodiment may, for instance, be used to play a known or predetermined audio sequence from the portable audio player 130 via said earphone 120, while simultaneously recording an EEG signal received via brain activity monitor 100. The EEG recording may, for instance, be made on the digital electronic storage device 260 that may be part of the portable audio player 130. In this way it may be possible to catalogue the music by the effects it produces on the subject.

In one embodiment of the invention, the digital filtering circuit 350 may be used to simply classify the EEG into being either below 13 Hz, i.e., either Alpha waves, indicative of a relaxed state, or above 13 Hz, i.e., Beta waves, indicative of being alert. The output may, for instance, be simply show up as one of two colors, such as, but not limited to, red or green. In this way a user may catalogue their music into that which makes them alert and that which makes them relax.

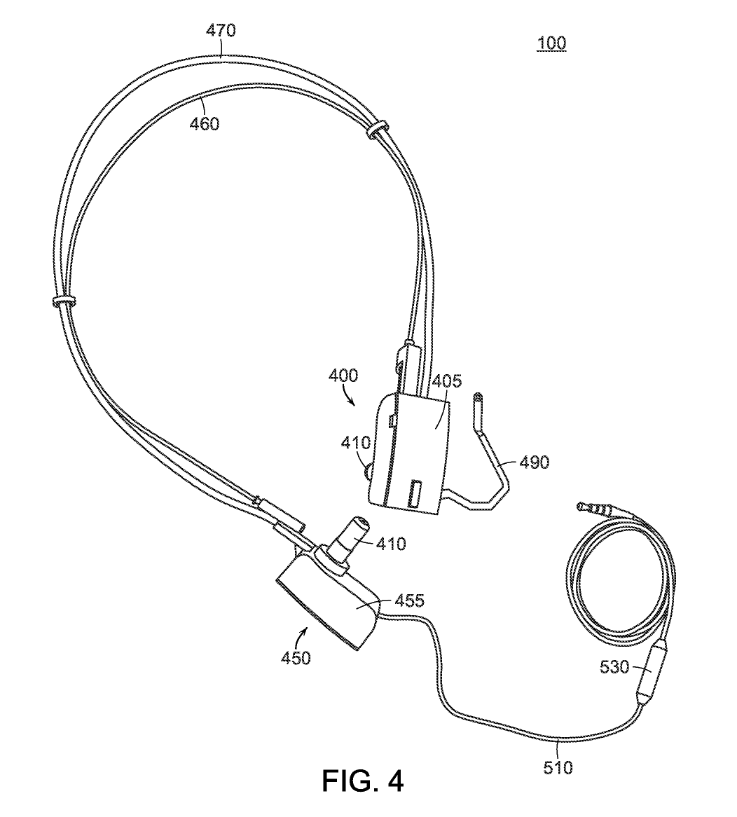

FIGS. 4-7 illustrate a third preferred embodiment of the brain activity monitor. It should be noted that not all features are described for this embodiment, especially the features already shown in the two embodiments shown above. As long as the features of the first and second embodiments are not in contradiction with the following descriptions of the third embodiment, they should be considered included.

FIG. 4 shows a top perspective view of a third preferred embodiment of the present invention. Shown in FIG. 4 is the brain activity monitor 100 having a first recording unit 400 having a first recording uniting casing 405 and a second recording unit 450 having a second recording unit casing 455. The first recording unit 400 is connected with the second recording unit 450 with a connecting cable 460 and a structural support cable 470. There is an EEG electrode 410 mounted on the second recording unit casing 455. A similar EEG 410 is mounted on the first recording unit casing 405. In addition, an earth cable 490 is mounted on the first recording unit casing 405.

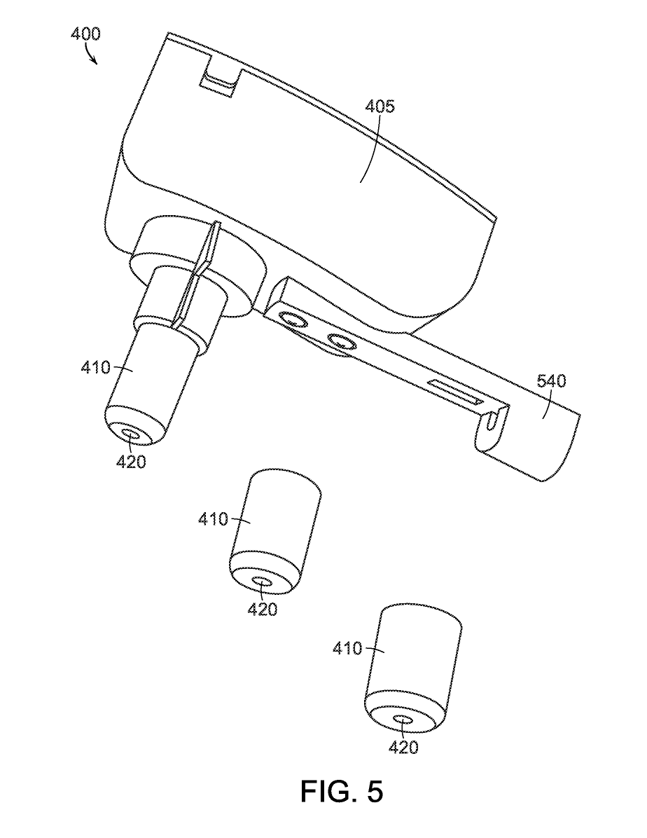

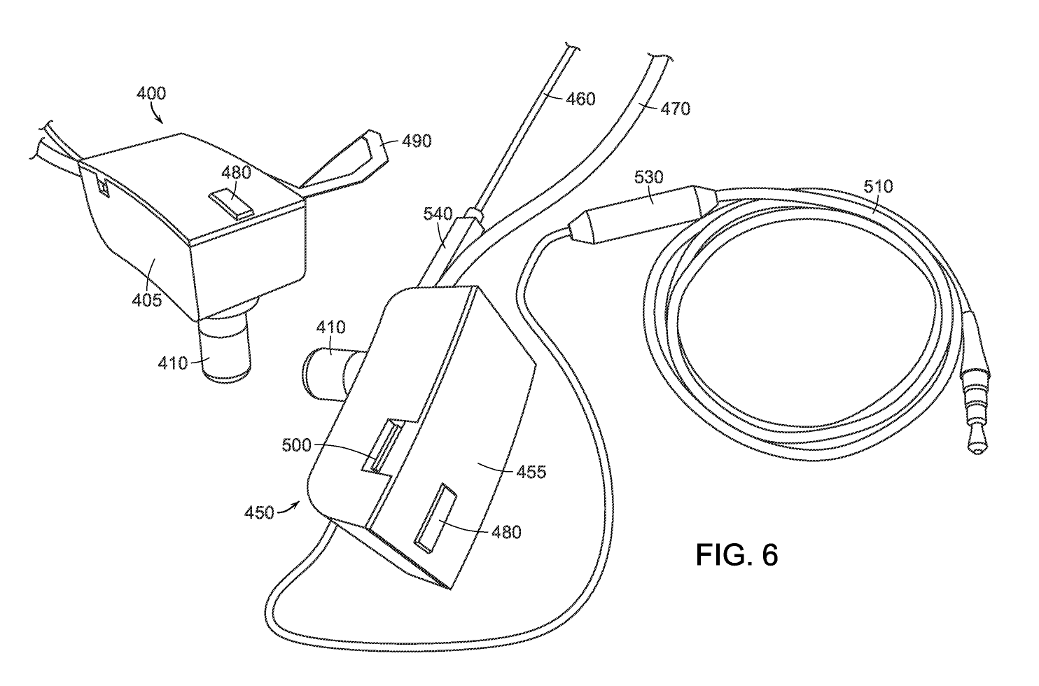

FIG. 5 shows a front perspective view of a single brain activity recording unit of the third preferred embodiment with the exchangeable EEG electrodes having different sizes. Shown in FIG. 5 is the first brain activity recording unit 400 having a first recording unit casing 405. Three EEG electrodes 410 are also shown in FIG. 5, with one EEG electrode 410 being mounted on the first recording unit casing 405. Each EEG electrode 410 has an earphone outlet 420 on the top end, as depicted in FIG. 5. Also shown in FIG. 5 is a cable support 540 connected to the first recording unit casing 405.

As shown in FIG. 4, the third embodiment of the brain activity monitor preferably has two recording units. Preferably, the two recording units have similar casings that may be used to house circuitry and power sources. The first and second recording unit may house different components. However, it should be noted that such components are all functionally connected and putting one particular component in one particular case is just a matter of industrial convenience. In describing the current embodiments, particular parts may be illustrated as housed in or attached to a particular recording unit, but it should be clear that the parts may be repositioned and such changes will not affect the overall design of the invention.

As indicated above, the brain activity monitor 100 may have two recording units, which is preferred. However, the brain activity monitor 100 may also only have one functioning recording unit with one recording unit housing, as shown in FIG. 5. All the previous discussions regarding the inner structures of the brain activity monitor still apply here. The third preferred embodiment may include a low-noise EEG sensor having an EEG electrode 430, an earphone, and an electronic processing circuitry, as shown in the first and second embodiments. The electronic processing circuitry may include an analogue amplifier designed to amplify electrical signals in a range of 0.5 Hz to 100 Hz, and an analogue-to-digital conversion circuit and a digital filtering circuit, as described above for the second embodiment. The electronic processing circuitry and the earphone may be housed in the recording unit casing.

As shown in FIG. 4, the brain activity monitor 100 may include an audio cable 510. The audio cable 510 may have an audio control 530. The brain activity monitor 100 may be connected, via the audio cable 510, to an audio player including but not limited to MP3 players such as iPod®, CD players, radios, personal computers such as desktop and laptop PC, and portable computing devices such as tablet PCs and smart phones, including but not limited to iPhone® and Android® phones. The audio control 530 may be used to control the volume of the audio signals.

The low-noise EEG sensor 140 may comprise an EEG electrode 410 shaped and sized to fit in a mammalian auditory canal. FIG. 5 shows a preferred cylindrical shape for the EEG electrode 410. However, other shapes may also be used such as a tapered cylinder. In addition, since the auditory canals from different individuals are generally different in size, it is preferred to have several EEG electrodes 410 with different length and diameter available, as shown in FIG. 5. The EEG electrodes are exchangeable, making the device more versatile in its usage.

As shown in FIG. 4, a recording unit may also include an earth cable 490. The earth cable, together with the EEG electrode 410, is considered a part of the low-noise EEG sensor. The earth cable 490 is shaped and sized in such a manner that it easily stays in contact with a user's ear when the electrode 410 is inserted into the user's auditory canal. The earth cable 490 may be mounted on either the first recording unit housing 405 or the second recording unit housing 455. Preferably, there is only one earth cable 490.

The size of the brain activity monitor is preferably small and portable. The preferred dimension of each recording unit casing is between 1-20 centimeters. In addition, each of the recording unit casing is preferred to be less than 1 kg, with the preferred weight between 5-500 g. The casings are preferred to be strong and durable, made from materials such as but not limited to metal, glass or fiberglass, wood, plastics, or any combination there of.

FIG. 6 shows a front perspective view of the third preferred embodiment with more details. Shown in FIG. 6 is the brain activity monitor 100 having a first recording unit 400 having a first recording uniting casing 405 and a second recording unit 450 having a second recording unit casing 455. The first recording unit 400 is connected with the second recording unit 450 with a connecting cable 460 and a structural support cable 470. There are EEG electrodes 410 mounted on the first recording unit casing 405 and second recording unit casing 455. In addition, an earth cable 490 is mounted on the first recording unit casing 405. There are power indicators 480 on the first recording unit casing 405 and second recording unit casing 455. Also shown in FIG. 6 is an audio cable 510, which may serve to connect the brain activity monitor 100 to an audio player. Moreover, there is a USB port 500 on the second recording unit 450.

It is preferred that the brain activity monitor include a power source to sustain the detection, recordation, and transfer of signals. The power source is functionally connected to the low-noise EEG sensor and the processing circuitry. The power source may be any kind of energy generating or energy storing devices. Preferably, the power source may be one or more batteries, such as the regular AAA zinc-carbon or alkaline battery, or any other type or size that may fit the needs of the monitor in terms of energy consumption, heat tolerance, and/or physical accommodation. The battery may be disposable or rechargeable, with the preferred form to be rechargeable. The types of batteries to be used as the power source include but are not limited to: lithium batteries, zinc-carbon batteries, alkaline batteries, aluminum batteries, dry-cell batteries, lead-acid batteries, nickel batteries, potassium batteries, and sodium-ion batteries. The power source is preferably housed in the recording unit casing.

The connecting cable 460, as shown in FIG. 4 and FIG. 6, may serve multiple functions. It may include sub-cables that may serve as common ground line, transfer audio signals, and transfer the encoded EEG signals. It may also include another sub-cable for power supply to the recording unit where the power source is not residing. The structural support cable 470 helps to sustain the physical integrity of the multi-unit design and provide comfort when the units are being worn on a subject's head.

As indicated above in FIG. 6, the brain activity monitor may include power indicators 480, which may serve to signal the power status of the device—whether it is on or off. Preferably, the power indicator is an embedded LED light or lights that are connected to the power source. When the power source is properly connected to the other components of the monitor, the power indicators 480 light up. Alternatively, the power indicator 480 may use audio signals to indicate the power status. The power indicator 480 is preferred to be mounted to the recording unit housings. It is possible to have one, two, or multiple power indicators 480.

Preferably, the brain activity monitor 100 may include at least one switch to control and possibly adjust the power supply of the device. The switch is connected to the power supply, the low-noise EEG sensor, and the processing circuitry. The switch may be a separate component. Alternatively, the power indicators 480 may also serve as power switches as shown in FIG. 6. The power indicator 480 may be pressed to control the power supply to the monitor.

To charge the rechargeable battery, if one is included, there are multiple ways. The third preferred embodiment has a USB port 500 positioned on one of the recording units. The USB technology is generally known in the art and it may be used to provide power and transfer data. The monitor may be connected to a PC or a portable computing device to charge the battery in the monitor and/or transfer and exchange data between the monitor and the PC or the portable computing device. The USB port 500 is where a USB cable may be plugged.

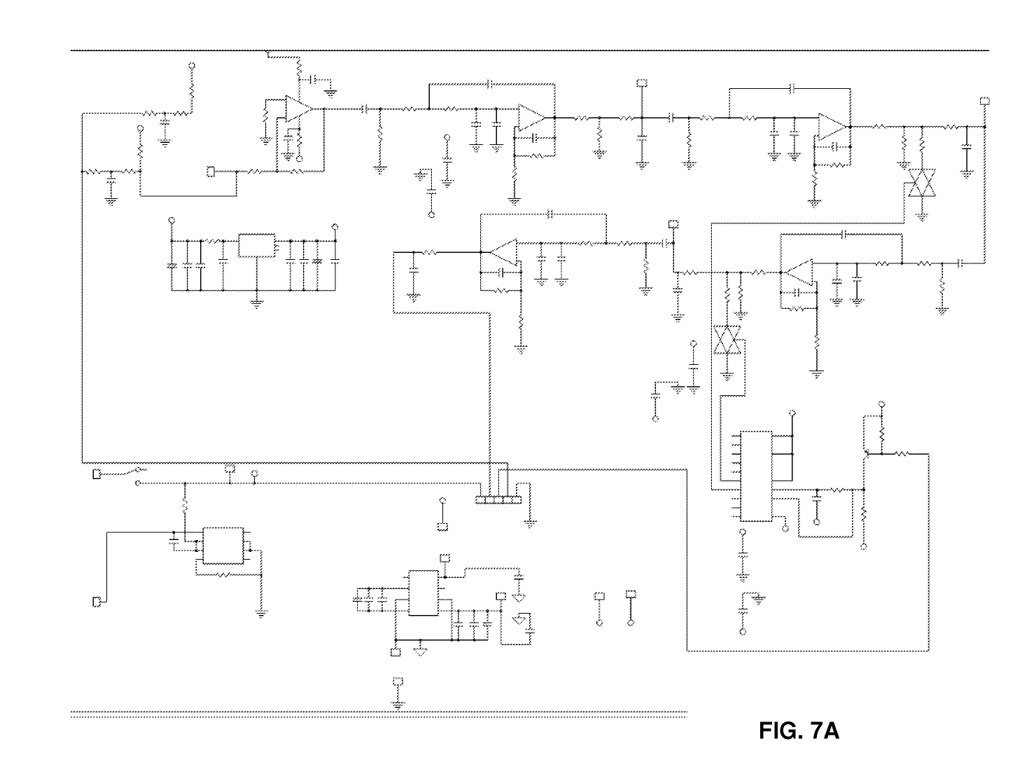

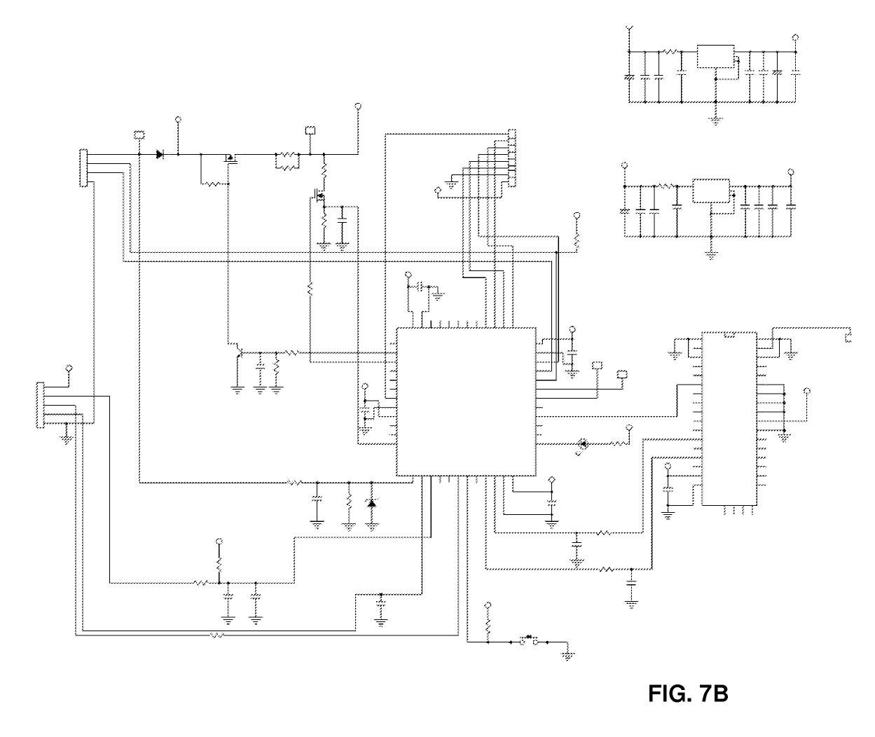

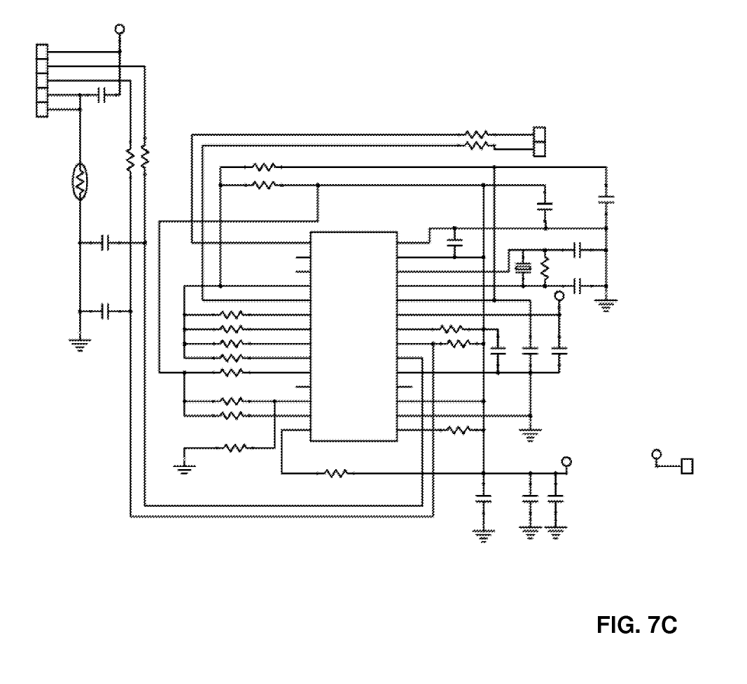

FIG. 7A shows a first circuit diagram of the third preferred embodiment of the present invention.

FIG. 7B shows a second circuit diagram of the third preferred embodiment of the present invention.

FIG. 7C shows a third circuit diagram of the third preferred embodiment of the present invention.

The brain activity monitor in general may be used to detect and record the EEG signals from the subject's brains. With the third embodiment in particular, the EEG electrodes 410 may be inserted into the auditory canals of the subject. The monitor as a whole may be worn on a subject's head, with the stabilization and support from the structural support cable 470. The audio cable 510 may be plugged into an audio player. Sound may come out of the audio outlet 420 on the EEG electrode 410. The low-noise EEG sensor, including the EEG electrode 410 and the earth cable 490, detects and transfers the EEG signals to the processing circuitry, which is housed in the recording units. The monitor includes a rechargeable battery as the power source. A USB port may be used to charge the battery and/or transfer data. In addition, there are power indicators 480 on the recording units to signal the power status of the monitor. Such power indicators may also serve as switches that control the power supply.

The recorded EEG signals are transferred to a digital storage device. The transfer may be made with any suitable connections such as, but not limited to, a wired connection, an optical connection, a wireless connection, a WiFi connection or a BlueTooth® connection, or some combination thereof. The preferred means is the BlueTooth® connection. The digital storage device may be the same as the audio player or may be separate apparatus. Preferably, the digital storage device and the audio player refer to the same device.

The current EEG monitoring device may be used to monitor brain signals and to record and transfer such signals to a processing and recording device. The music and the brain signals may be processed to decipher (by unique algorithms) the linking of these two sources by examining the alpha/beta patterns of the brain signals. The resulted analysis may be documented and shared between users of the EEG device and therapists.

The current invention has undergone extensive testing and the functionality of different parts underwent rigorous examination. The third embodiment has been tested on PCs, tablet PCs, and smart phones such as Android® devices. The signals detected and recorded are strong and clear.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A brain activity monitor, comprising:
an earphone functionally connected to an audio player;
a low-noise EEG sensor having at least one EEG electrode shaped and sized to fit within a mammalian auditory canal, and an electronic processing circuitry in close proximity to said EEG electrode, said processing circuitry comprising an analogue amplifier designed to amplify electrical signals within a range of 0.5 Hz to 100 Hz, an analogue-to-digital conversion circuit and a digital filtering circuit; and
an earth cable having an ear attachment device functionally attached to said low-noise EEG sensor;
wherein said low-noise sensor is functionally connected to a portable digital electronic storage device.

2. The brain activity monitor of claim 1, wherein said EEG electrode is a dry-contact electrode.

3. The brain activity monitor of claim 1, wherein said electronic processing circuitry is located in same mammalian auditory canal as said EEG electrode.

4. The brain activity monitor of claim 3, wherein said earphone is located in same mammalian auditory canal.

5. The brain activity monitor of claim 4, wherein said portable digital electronic storage device is a part of said portable audio player.

6. The brain activity monitor of claim 1, wherein said digital filtering circuit includes a 60 Hz notch filter.

7. The brain activity monitor of claim 1, wherein said analogue-to-digital conversion circuit is removably clipped to a user's ear-lobe.

8. The brain activity monitor of claim 1, wherein said analogue-to-digital conversion circuit is an 18 bit AD convertor.

9. The brain activity monitor of claim 1, wherein said dry-contact electrode is a capacitance electrode.

10. The brain activity monitor of claim 1, wherein said EEG electrode is a silicon needle electrode.

11. A method of analyzing brain response to audio input, comprising:
providing a an earphone functionally connected to a portable audio player;
providing a low-noise EEG sensor having at least one EEG electrode shaped and sized to fit within a mammalian auditory canal, and electronic processing circuitry in close proximity to said EEG electrode, said processing circuitry comprising an analogue amplifier designed to amplify electrical signals in a range of 0.5 Hz to 100 Hz, an analogue-to-digital conversion circuit and a digital filtering circuit;
providing an earth cable having an ear attachment device wherein said earth cable and said low-noise sensor are both functionally connected to a portable digital electronic storage device; and
substantially simultaneously playing an audio sequence from said portable audio player via said earphone, recording an EEG signal received via said dry-contact electrode on said portable digital electronic storage device.

12. A brain activity recording unit comprising:
a recording unit casing;
an earphone functionally connected to an audio player;
a low-noise EEG sensor comprising:
at least one EEG electrode shaped and sized to fit within a mammalian auditory canal,
and an earth cable;
and an electronic processing circuitry functionally connected to said EEG electrode, said processing circuitry comprising:
an analogue amplifier designed to amplify electrical signals within a range of 0.5 Hz to 100 Hz,
an analogue-to-digital conversion circuit,
and a digital filtering circuit;
wherein
said processing circuitry is housed in the recording unit casing,
and said EEG electrode and said earth cable are mounted on the recording unit casing.

13. The brain activity recording unit of claim 12, further comprising
an audio cable connecting the earphone to the audio player.

14. The brain activity recording unit of claim 12, further comprising
a power source functionally connected to the low-noise EEG sensor and to the processing circuitry.

15. The brain activity recording unit of claim 14, wherein
the power source is a rechargeable battery.

16. The brain activity recording unit of claim 15, further comprising:
a USB port functionally connected to the rechargeable battery and the processing circuitry.

17. The brain activity recording unit of claim 12, wherein
the EEG electrode has an audio outlet.

18. The brain activity recording unit of claim 14, further comprising:
a switch controlling the connection of the power source to the low-noise EEG sensor and the processing circuitry.

19. The brain activity recording unit of claim 14, further comprising
a power indicator functionally connected to the power source, wherein
the power indicator signals power status of the power source.

20. The brain activity recording unit of claim 19, further comprising
a switch controlling the connection of the power source to the low-noise EEG sensor and the processing circuitry, wherein
the switch and the signal indicator are integrated.

21. A brain activity monitor, comprising:
a low noise EEG sensor functionally connected to a digital electronic storage device,
a first recording unit, and
a second recording unit; wherein
said first recording unit comprises:
a first EEG electrode, said first EEG electrode functionally connected to the low noise EEG sensor and being shaped and sized to fit within a mammalian auditory canal, and being mounted on a first recording unit casing,
a first earphone functionally connected to an audio player,
and a first electronic processing circuitry functionally connected to said first EEG electrode, said first processing circuitry comprising:
an analogue amplifier capable to amplify electrical signals within a range of 0.5 Hz to 100 Hz,
an analogue-to-digital conversion circuit, and a digital filtering circuit;
wherein said first processing circuitry is housed in the first recording unit casing; and
said second recording unit comprising:
a second EEG electrode shaped and sized to fit within a mammalian auditory canal, and being functionally connected to the low-noise EEG sensor, and being mounted on a second recording casing, and
a second earphone functionally connected to the audio player.

22. The brain activity monitor of claim 21, wherein
the low-noise EEG sensor further comprises an earth cable mounted on the first recording unit casing.

23. The brain activity monitor of claim 21, further comprising
a connecting cable connecting the second EEG electrode to the low-noise EEG sensor;
and a structural support cable connecting the first recording unit to the second recording unit.

24. The brain activity monitor of claim 21, further comprising
a rechargeable battery functionally connected to the low-noise EEG sensor and the processing circuitry.

25. The brain activity monitor of claim 24, wherein
the rechargeable battery is housed in the second recording unit casing.

26. The brain activity monitor of claim 24, further comprising
a USB port functionally connected to the rechargeable battery and the processing circuitry.

27. The brain activity monitor of claim 21, further comprising
digital electronic storage device connected to the low-noise EEG sensor and the processing circuitry.

28. The brain activity monitor of claim 24, further comprising
a switch controlling the connection of the rechargeable battery to the low-noise EEG sensor and the processing circuitry.

29. The brain activity monitor of claim 28, further comprising
a power indicator functionally connected to the rechargeable battery, wherein
the power indicator signals power status of the rechargeable battery.

30. The brain activity monitor of claim 28, further comprising
the processing circuitry, wherein
the switch and the signal indicator are integrated.
